(19) United States
(12) Patent Application Publication
(10) Pub. No.: US 2023/0310059 A1
(43) Pub. Date: Oct. 5, 2023

(54) POWER SUPPLY DEVICE AND POWER SUPPLY METHOD

(71) Applicant: Japan Lifeline Co., Ltd., Tokyo (JP)

(72) Inventors: Takuya HIRAO, Tokyo (JP); Kohei SAKAKI, Tokyo (JP); Makoto KATO, Tokyo (JP); Tomoharu KOISO, Tokyo (JP)

(21) Appl. No.: 18/069,440

(22) Filed: Dec. 21, 2022

(30) Foreign Application Priority Data
Mar. 29, 2022 (JP) 2022-054282

(51) Int. Cl. A61B 18/12

(52) U.S. Cl. CPC A61B 18/1206; A61B 2018/1467

(57) ABSTRACT

[Problem]

Provided is a power supply device and the like capable of improving convenience.

[Solution]

A power supply device according to an embodiment of the present disclosure includes: a power supply unit that supplies power to an electromedical device including a plurality of electrodes; and a control unit that controls the power supply unit such that the power is supplied to a part of the plurality of electrodes.

### TECHNICAL FIELD

The present disclosure relates to a power supply device and a power supply method.

### BACKGROUND ART

For example, Patent Literature 1 discloses an ablation system including an electromedical device, such as an ablation catheter, and a power supply device.

### CITATION LIST

### Patent Literature

Patent Literature 1: JP 2019-500170 T

### SUMMARY OF INVENTION

### Technical Problem

In the ablation system, improvement in convenience during ablation to an affected area has been demanded. It is desirable to provide a power supply device and a power supply method that allow improving convenience.

### Solution to Problem

A power supply device according to an embodiment of the present disclosure includes a power supply unit and a control unit. The power supply unit supplies power to an electromedical device including a plurality of electrodes. The control unit controls the power supply unit such that the power is supplied to a part of the plurality of electrodes.

A power supply method according to an embodiment of the present disclosure supplies power to an electromedical device including a plurality of electrodes. The power supply method includes: supplying the power to a part of the plurality of electrodes; and supplying the power to a remaining electrode among the plurality of electrodes.

### BRIEF DESCRIPTION OF DRAWINGS

FIG. 1 is a block diagram schematically representing an overall configuration example of an ablation system according to an embodiment of the present disclosure.

FIG. 2 is a schematic diagram illustrating a schematic configuration example of an ablation catheter illustrated in FIG. 1.

FIG. 3A is a schematic diagram for describing an ablation method according to a reference example.

FIG. 3B is a schematic diagram for describing an ablation method according to a comparative example.

FIG. 3C is a schematic view for describing an ablation method according to Example 1.

FIG. 4 is a timing diagram for describing the ablation method according to the comparative example.

FIG. 5 is a timing diagram for describing the ablation method according to Example 1.

FIG. 6 is a schematic diagram representing a configuration example of electrode groups applied to ablation methods according to Examples 2-1 and 2-2 in a modified example.

FIG. 7 is a timing diagram for describing the ablation method according to Example 2-1.

FIG. 8 is a timing diagram for describing the ablation method according to Example 2-2.

### DESCRIPTION OF EMBODIMENTS

An embodiment of the present disclosure will be described below in detail with reference to the drawings. Note that the description will be given in the following order.
1. Embodiment (Example of Ablation Using, for Example, Irreversible Electroporation Method)
2. Modified Examples (Example of Grouping into a plurality of Electrode groups and Ablation)
3. Other Modified Examples

### 1. Embodiment

### Configuration of Ablation System 5

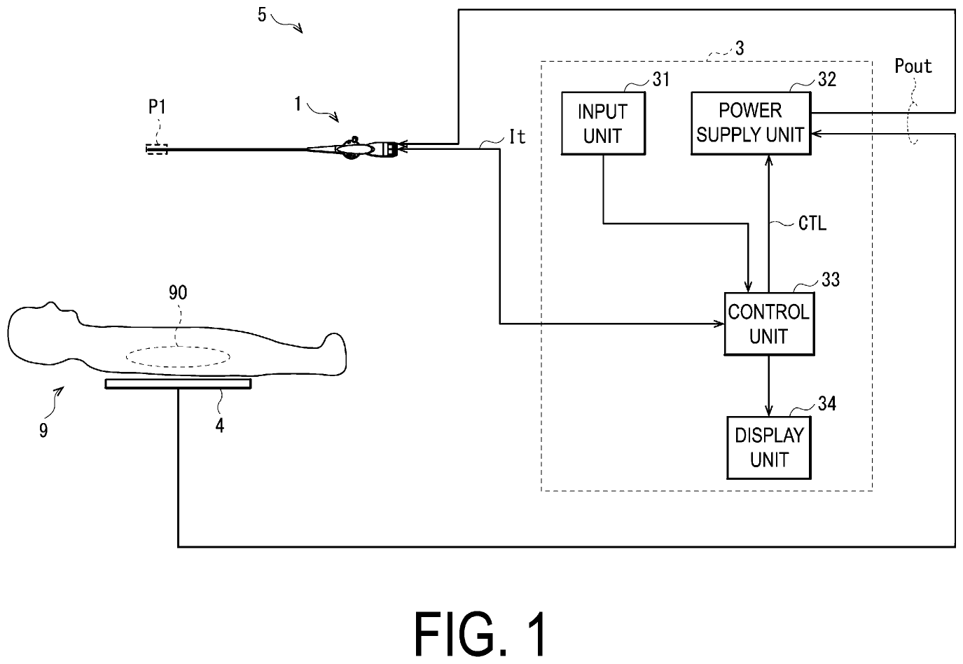

FIG. 1 schematically illustrates an overall configuration example of an ablation system 5 according to an embodiment of the present disclosure in a block diagram. The ablation system 5 is a system used when an affected area 90 in a body of a patient 9 is treated, and performs predetermined ablation on the affected area 90.

Note that the affected area 90 includes, for example, an affected area having, for example, an arrhythmia, and an affected area having a tumor, such as a cancer (for example, liver cancer, lung cancer, breast cancer, kidney cancer, and thyroid cancer).

The ablation system 5 includes an ablation catheter 1 and a power supply device 3. In the case of ablation using the ablation system 5, for example, a counter electrode plate 4 illustrated in FIG. 1 is also appropriately used. Since a “power supply method” in the present disclosure is embodied in the ablation system of the present disclosure, the method will be described together below.

### Ablation Catheter 1

The ablation catheter 1 is an electrode catheter that is inserted into the body of the patient 9 through a blood vessel and ablates the affected area 90 to treat, for example, an arrhythmia. The ablation catheter 1 may have an irrigation mechanism in which a predetermined fluid (for example, an irrigation fluid (liquid) such as physiological saline) is discharged (injected) at the time of ablation.

The ablation catheter 1 corresponds to a specific example of an “electromedical device” in the present disclosure.

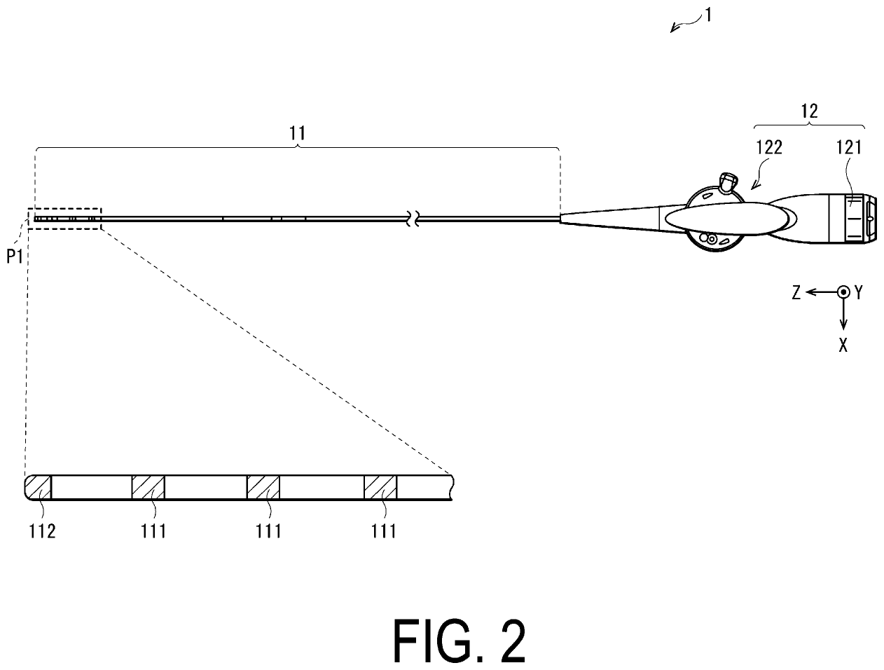

FIG. 2 schematically represents a schematic configuration example of the ablation catheter 1. The ablation catheter 1 includes a shaft 11 (catheter shaft) as a catheter body and a handle 12 attached to the base end of the shaft 11.

The shaft 11 has a flexible tubular structure (tubular member) and extends along its axial direction (Z-axis direction). The shaft 11 is made of, for example, a synthetic resin such as polyolefin, polyamide, polyether polyamide, or polyurethane. The shaft 11 has a so-called single-lumen structure in which one lumen (pore, through-hole) is formed. Alternatively, the shaft 11 has a so-called multi-lumen structure in which a plurality of (for example, four) lumens are formed. Along an axial direction of the shaft 11, both of a region having a single-lumen structure and a region having a multi-lumen structure may be provided. Each of various thin wires (for example, lead wires or operating wires) (not illustrated) are inserted into the lumen in a mutually electrically insulated state.

A mechanism (temperature measuring mechanism) for measuring the temperature close to a distal end P1 (around the affected area 90) is provided close to the distal end P1 of the shaft 11. Information indicating the measured temperature close to the distal end P1 (temperature information It) is supplied from the ablation catheter 1 to the power supply device 3 (a control unit 33 described later) (see FIG. 1).

As illustrated in the enlarged view close to the distal end P1 in FIG. 2, a plurality of electrodes 111 and 112 are provided close to the distal end P1 of the shaft 11. Specifically, in the example of FIG. 2, the three ring-shaped electrodes (electrodes 111) and one distal end electrode (electrode 112) are disposed at predetermined intervals along the axial direction (Z-axis direction) of the shaft 11. Although the details will be described later, ablation is performed by energization between the plurality of electrodes 111 and 112 and the counter electrode plate 4.

Each of the electrodes 111 is fixedly disposed on the outer peripheral surface of the shaft 11. On the other hand, the electrode 112 is fixedly disposed at the most distal end of the shaft 11. Each of the electrodes 111 and 112 is electrically connected to the handle 12 via a plurality of the lead wires inserted into the lumen of the shaft 11. Each of the electrodes 111 and 112 is constituted by a metal material having good electrical conductivity, such as aluminum (Al), copper (Cu), stainless steel (SUS), gold (Au), or platinum (Pt). In order to improve the contrast with respect to X-rays when the ablation catheter 1 is used, the electrodes are preferably made of platinum or an alloy thereof.

The handle 12 is attached to the base end of the shaft 11 and includes a handle body 121 (grip portion) and a rotating operating unit 122.

### Power Supply Device 3

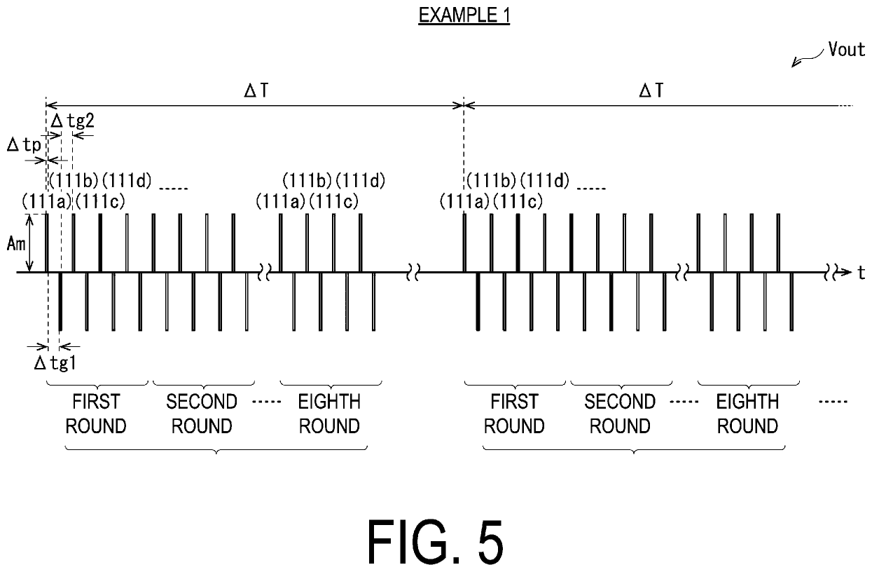

The power supply device 3 is a device that supplies power Pout for ablation between the electrodes 111 and 112 in the ablation catheter 1 and the counter electrode plate 4. That is, the power supply device 3 supplies the power Pout to the ablation catheter 1. Details of the power Pout (such as a waveform example) will be described later (FIG. 5). As illustrated in FIG. 1, the power supply device 3 includes an input unit 31, a power supply unit 32, the control unit 33, and a display unit 34.

The input unit 31 is a unit for inputting various setting values and instruction signals (operation signals) for instructing a predetermined operation. Examples of various setting values include setting power of the power Pout and various thresholds. The operation signal is input from the input unit 31 in response to an operation by an operator (for example, a technician or the like) of the power supply device 3. However, various setting values may be set in the power supply device 3 in advance, for example, when the product is shipped, rather than being input in response to the operation by the operator. The setting value input by the input unit 31 is supplied to the control unit 33. The input unit 31 includes, for example, a predetermined dial, buttons, and a touch panel.

The power supply unit 32 outputs the power Pout according to a control signal CTL supplied from the control unit 33. The power supply unit 32 includes a predetermined power supply circuit (for example, a switching regulator).

The control unit 33 controls the entire power supply device 3, performs predetermined arithmetic processing, and is constituted using, for example, a microcomputer. As illustrated in FIG. 1, for example, the control unit 33 controls the supply operation of the power Pout in the power supply unit 32 using the control signal CTL.

The display unit 34 is a unit (monitor) that displays various pieces of information and outputs these pieces of information to the outside. The display unit 34 is configured using a display according to various modes (for example, a liquid crystal display, a cathode ray tube (CRT) display, or an organic electro luminescence (EL)).

### Counter Electrode Plate 4

As illustrated in FIG. 1, for example, the counter electrode plate 4 is used in a state of being attached to the body surface of the patient 9 at the time of ablation. At the time of ablation, energization is performed (the power Pout is supplied) between the electrodes 111 and 112 of the ablation catheter 1 and the counter electrode plate 4.

### Operation and Advantages and Effects

### A. Basic Operation

In the ablation system 5, the distal end P1 of the shaft 11 of the ablation catheter 1 is inserted into the body of the patient 9 through a blood vessel during treatment of, for example, an arrhythmia.

Then, the power Pout is supplied from the power supply device 3 between the electrodes 111 and 112 close to the distal end P1 of the shaft 11 and the counter electrode plate 4, thus performing ablation on the affected area 90 inside the body of the patient 9. The energization at the time selectively ablates a treatment target site (procedure part) in the patient 9, and transvascular treatment of, for example, an arrhythmia is performed. Specifically, the ablation is performed using, for example, high frequency ablation (Radio Frequency Ablation: RFA) or Pulsed electric Field Ablation (PFA).

### B. Details of Ablation Operation

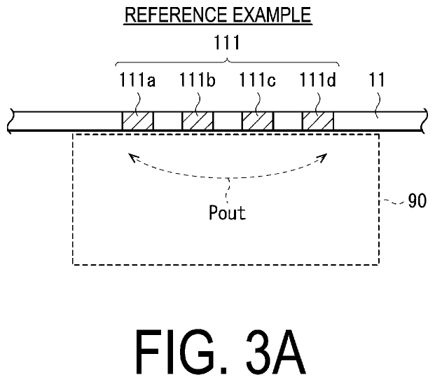

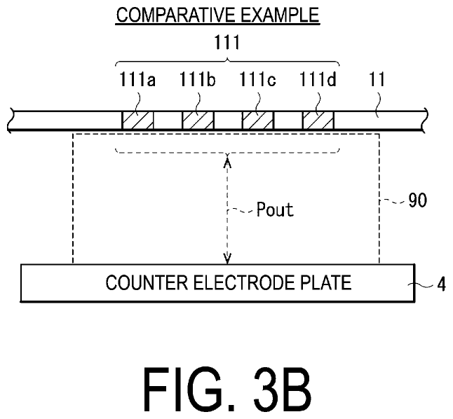

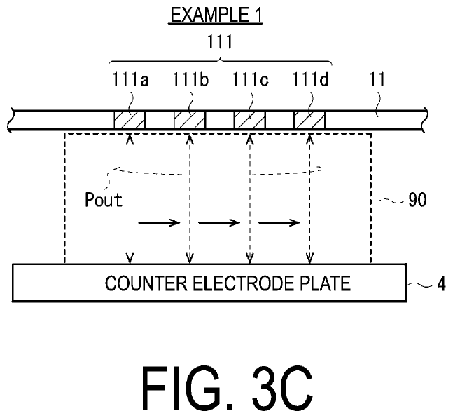

Next, with reference to FIGS. 3A to 3C, 4, and 5, details of the ablation operation (ablation method) according to the present embodiment will be described with comparison to a reference example and a comparative example.

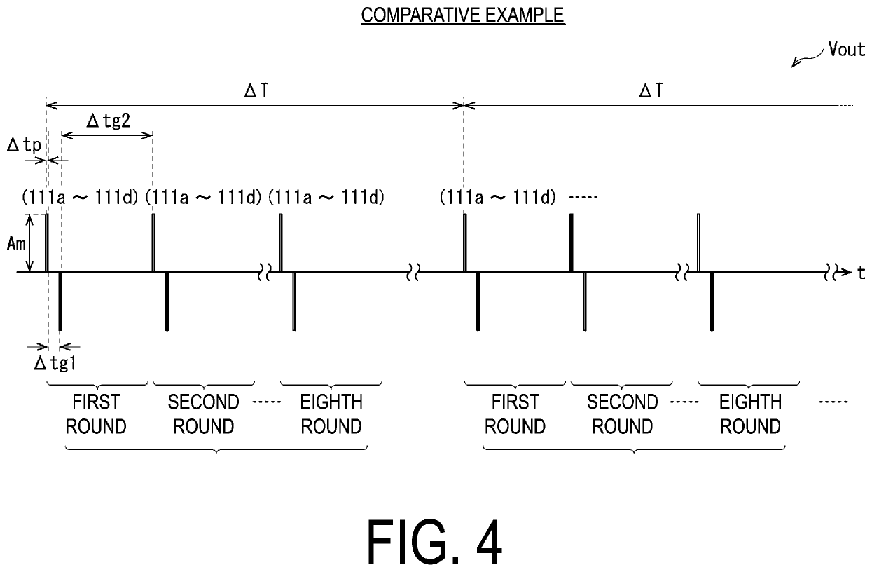

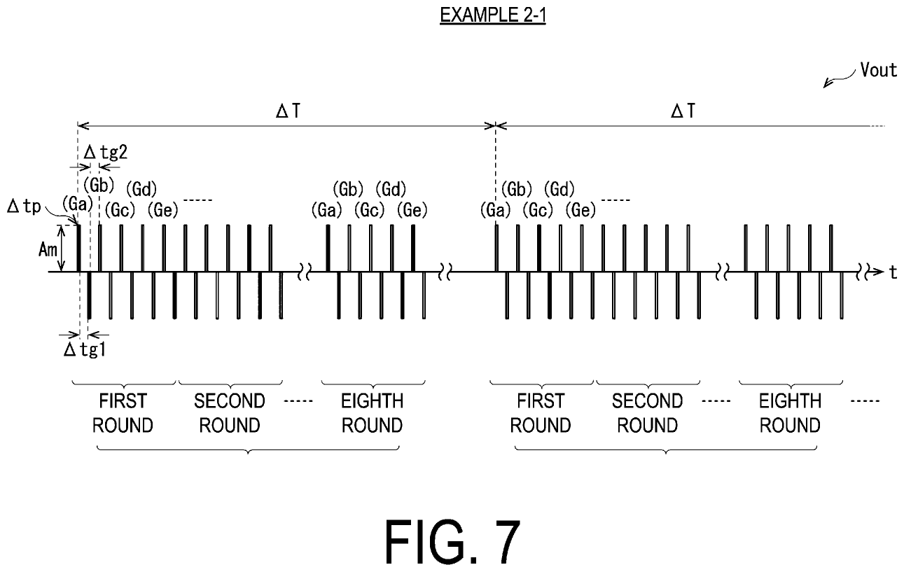

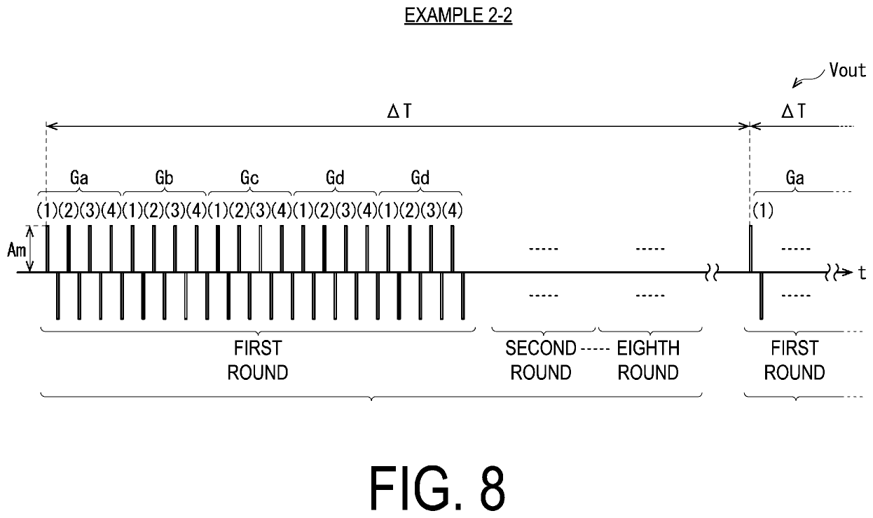

FIG. 3A is a schematic diagram for describing the ablation method according to the reference example, and FIG. 3B is a schematic diagram for describing the ablation method according to the comparative example. FIG. 3C is a schematic diagram for describing the ablation method according to an example (Example 1) of the present embodiment. FIG. 4 is a timing chart for describing the ablation method according to the comparative example illustrated in FIG. 3B. FIG. 5 is a timing diagram for describing the ablation method according to Example 1 illustrated in FIG. 3C. In FIGS. 4 and 5, the horizontal axis indicates a time t, and the vertical axis indicates a voltage (potential difference from a reference potential). This point is also similar in FIGS. 7 and 8, which will be described later.

Note that all of the examples illustrated in FIGS. 3A to 3C (the reference example, the comparative example, and Example 1) are examples in which ablation is performed on the affected area 90 using the four electrodes 111 (electrodes 111a to 111d) in the shaft 11 for convenience. Also, all of the examples illustrated in FIGS. 4 and 5 are examples of the PFA (ablation using irreversible electroporation method) described above.

### B-1. Reference Example

First, in the ablation method of the reference example illustrated in FIG. 3A, the counter electrode plate 4 is not used, and the power Pout is supplied between the plurality of electrodes 111a to 111d on the shaft 11 (see the dashed line arrow in FIG. 3A), and thus the ablation on the affected area 90 is performed. That is, the ablation method of the reference example is ablation in a so-called bipolar type.

In the ablation method of this reference example, the range of ablation on the affected area 90 is relatively deep. Specifically, in the ablation method of the reference example, the range of ablation is, for example, a depth of approximately 2.1 [mm].

### B-2. Comparative Example

On the other hand, in the ablation method of the comparative example illustrated in FIG. 3B, the power Pout is supplied between the plurality of electrodes 111a to 111d on the shaft 11 and the counter electrode plate 4 (see the dashed arrow in FIG. 3B), and thus the ablation is performed on the affected area 90. That is, the ablation method of the comparative example is an ablation in a so-called monopolar type.

Specifically, as indicated by the dashed line in FIGS. 3B and 4, for example, in the ablation method of this comparative example, the power Pout is collectively supplied to all of the plurality of electrodes 111 (111a to 111d). Also, for example, as illustrated in FIG. 4, a voltage is alternately applied to both the positive side and the negative side with respect to the reference potential in a pulse waveform having a predetermined amplitude value Am (for example, approximately from 100 to 3000 [V]) and a predetermined pulse width Δtp (for example, approximately from 1 to 10 [μs]). Note that, each of time intervals Δtg1 and Δt2 between the pulse waveforms illustrated in FIG. 4 is approximately Δtg1=from 1 to 10 [μs] and Δtg2=from 1 to 100 [μs] as an example. Then, in the example of FIG. 4, the pulse waveforms on the positive side and the negative side with respect to the reference potential are repeated within a predetermined cycle ΔT (for example, approximately from 0.1 to 1 [s]) from the first round to the eighth round, and the ablation operation is repeated with the cycle ΔT as a unit (for example, the number of repetitions of approximately from 10 to 1000 times).

In the ablation method of this comparative example (in a monopolar type), the range of ablation on the affected area 90 is shallower than that of the ablation method of the reference example described above (in a bipolar type). Specifically, in the ablation method of the comparative example, the range of ablation is, for example, a depth of approximately 0.7 [mm]. Therefore, in the ablation method of the comparative example, convenience during ablation may be impaired.

### B-3. Example 1

In contrast, in the ablation method of Example 1 illustrated in FIG. 3C, the power Pout is supplied between the plurality of electrodes 111a to 111d on the shaft 11 and the counter electrode plate 4 (see the dashed arrow in FIG. 3C), and when the ablation is performed on the affected area 90, the following occurs. That is, when ablation is performed in the monopolar type similarly to the ablation method of the comparative example described above, in Example 1, the power Pout is supplied to a part of the plurality of electrodes 111 (111a to 111d), unlike the comparative example. In other words, the control unit 33 in the power supply device 3 controls the power supply unit 32 such that the power Pout is supplied to a part of the plurality of electrodes 111 when the ablation is performed. The control unit 33 controls the power supply unit 32 such that the power Pout is supplied to the remaining electrodes 111 among the plurality of electrodes 111. Note that the “remaining electrodes” at this time may be a part of or all of the plurality of electrodes 111.

Specifically, for example, as indicated by the arrows in FIGS. 3C and 5, in the ablation method of Example 1, the power Pout is sequentially supplied to a part of (one in this example) the plurality of electrodes 111. That is, for example, as illustrated in FIG. 5, the power Pout is sequentially supplied in the order of (electrode 111a→electrode 111b→electrode 111c→electrode 111d), and this sequential supply of the power Pout is repeated. Note that the order at the time of sequential supply may be, for example, a predetermined random order, not the arrangement order of the electrodes 111a to 111d, and the same applies to the modified examples described below (Examples 2-1 and 2-2).

In detail, for example, as illustrated in FIG. 5, a voltage is alternately applied to both of the positive side and the negative side with respect to the reference potential in a pulse waveform having the predetermined amplitude value Am (for example, approximately from 1000 to 3000 [V]) and the predetermined pulse width Δtp (for example, approximately from 1 to 10 [μs]). Note that, each of the time intervals Δtg1 and Δt2 between the pulse waveforms illustrated in FIG. 5 is approximately Δtg1=from 1 to 10 [μs] and Δtg2=from 1 to 10 [μs] as an example. Then, in the example of FIG. 5, the pulse waveforms on the positive side and the negative side with respect to the reference potential are repeated within the predetermined cycle ΔT (for example, approximately from 0.1 to 1 [s]) from the first round to the eighth round, and the ablation operation is repeated with the cycle ΔT as a unit (for example, the number of repetitions of approximately from 10 to 1000 times).

In the ablation method of Example 1 (in a monopolar type), the range of ablation on the affected area 90 is deeper than that of the ablation method of the comparative example described above (in a monopolar type). Specifically, in the ablation method of Example 1, the range of ablation is, for example, a depth of approximately 2.7 [mm]. That is, in the ablation method of Example 1, even in the monopolar type same as the comparative example, a depth equal to or greater than that of the ablation method of the reference example (a bipolar type) is ensured.

This is possibly caused because of the following. In the ablation method of the comparative example, since the power Pout is collectively supplied to all of the plurality of electrodes 111, as a result of a current during the ablation being dispersed and flowing in the plurality of electrodes 111, the current density during ablation decreases. In other words, in contrast, in the ablation method of Example 1, since the power Pout is (selectively) supplied to a part of the plurality of electrodes 111, as a result of the current during the ablation concentratively flowing to a part of the plurality of electrodes 111, compared to the case of the comparative example, the current density during the ablation increases. In this way, in Example 1, compared to the comparative example, the range of ablation on the affected area 90 is considered to be deeper than the case of a monopolar type.

### C. Advantages and Effects

Thus, in the present embodiment, when ablation is performed by energization between the plurality of electrodes 111 and the counter electrode plate 4, the power Pout is supplied to a part of the plurality of electrodes 111, and thus, the following occurs. That is, as described above, the range of ablation on the affected area 90 is deeper than the case of the comparative example in the case of a monopolar type as well. As a result, with the present embodiment, convenience can be improved during ablation on the affected area 90.

In the present embodiment, when the ablation is performed, the power Pout is sequentially supplied to a part of the plurality of electrodes 111, and thus the range of ablation can be deepened close to the disposed region of each of the plurality of electrodes 111 as described above. As a result, convenience during ablation can be further improved.

### 2. Modified Examples

Next, modified examples of the above-described embodiment will be described. The same components as those in the embodiment are designated by the same reference numerals, and the description thereof will be omitted as appropriate.

### Configuration

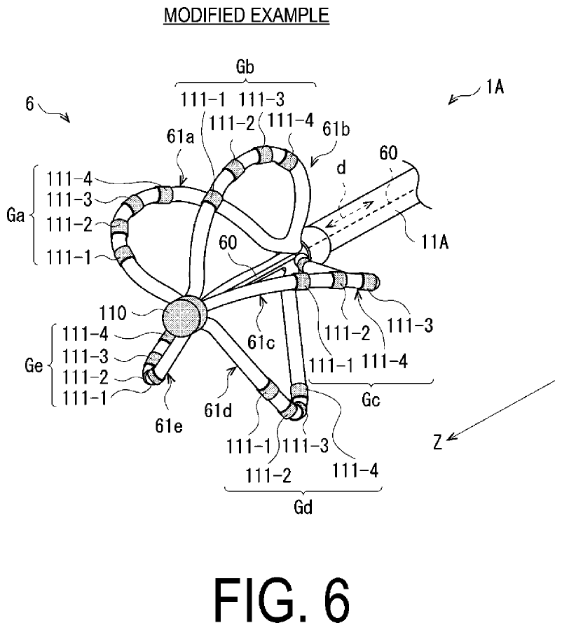

FIG. 6 schematically represents a configuration example of electrode groups Ga to Ge of an ablation catheter 1A applied to an ablation method according to the modified examples (Examples 2-1 and 2-2 described below).

The ablation catheter 1A of the modified example has a predetermined distal end proximal structure 6 close to the distal end of a shaft 11A. The distal end proximal structure 6 includes a branch point (a position at base end side of the distal end proximal structure 6) of the shaft 11A, a junction positioned close to the most distal end (close to a distal end tip 110) of the shaft 11A, and a plurality (five in this example) of branch structures 61a to 61e that are portions to individually connect between the branch point and the junction in a curved shape. In each of the branch structures 61a to 61e, one or a respective plurality of ring-shaped electrodes (four electrodes 111-1 to 111-4 in the example of FIG. 6) are spaced apart to be disposed at predetermined intervals along the extension direction of the curved shape. Note that, hereinafter, for convenience, description will be given with the electrodes 111-1 to 111-4 collectively referred to as electrodes 111 as appropriate.

Note that, for example, a deformation wire 60 illustrated in FIG. 6 may be configured such that displacement of the deformation wire 60 in both directions along the axial direction (Z-axis direction) of the shaft 11A (see the dashed arrow d) changes (deforms) the shape (so-called a “basket shape”) of the distal end proximal structure 6. Incidentally, the “basket shape” means that, for example, as illustrated in FIG. 6, the shape formed by the plurality of branch structures 61a to 61e is a shape similar to a pattern of the curved shape formed on the surface of the basketball.

The ablation catheter 1A corresponds to a specific example of the “electromedical device” in the present disclosure.

### Operation and Advantages and Effects

### A. Details of Ablation Operation

Next, with reference to FIGS. 7 and 8, in addition to FIG. 6, the details of the ablation operation (the ablation methods according to Examples 2-1 and 2-2) according to the present modified example will be described.

FIG. 7 is a timing diagram for describing the ablation method according to Example 2-1. FIG. 8 is a timing diagram for describing the ablation method according to Example 2-2. Note that both of the examples illustrated in FIGS. 7 and 8 are examples of the PFA (ablation using an irreversible electroporation method) similarly to the case of FIGS. 4 and 5 described above.

First, in the ablation methods of Examples 2-1 and 2-2 as well, similarly to the ablation method of Example 1 described above, the power Pout is supplied to a part of the plurality of electrodes 111 (111-1 to 111-4). Specifically, as described in detail below, similarly to Example 1, in Examples 2-1 and 2-2 as well, the power Pout is sequentially supplied to a part of the plurality of electrodes 111, and the sequential supply of the power Pout is repeated.

Additionally, in both of the ablation methods of Examples 2-1 and 2-2, as illustrated in FIG. 6, the plurality of electrodes 111 included in the distal end proximal structure 6 are grouped into a plurality of the electrode groups (the five electrode groups Ga to Ge in this example). That is, the plurality of electrodes 111 (111-1 to 111-4) disposed for each of the plurality of branch structures 61a to 61e form each of the electrode groups Ga to Ge. Specifically, the electrode group Ga is configured by the four electrodes 111 disposed on the branch structure 61a, the electrode group GB is configured by the four electrodes 111 disposed on the branch structure 61b, and the electrode group Gc is configured by the four electrodes 111 disposed on the branch structure 61c. Similarly, the electrode group Gd is configured by the four electrodes 111 disposed on the branch structure 61d, and the electrode group Ge is configured by the four electrodes 111 disposed on the branch structure 61e.

### A-1. Example 2-1

For example, as illustrated in FIG. 7, in the ablation method of Example 2-1, the power Pout is sequentially supplied to each of the plurality of electrode groups Ga to Ge. That is, in this example, in the order of (electrode group Ga→electrode group Gb→electrode group Gc→electrode group Gd→electrode group Ge), the power Pout is sequentially supplied to the electrodes 111 in each of the electrodes Ga to Ge, and the sequential supply of the power Pout is repeated. In the ablation method of Example 2-1, when the power Pout is sequentially supplied to each of the plurality of electrode groups Ga to Ge, the power Pout is collectively supplied to at least a part of the electrodes 111 (all of the electrodes 111 included in each of the electrode groups Ga to Ge in this example) included in each of the electrode groups Ga to Ge.

In detail, for example, as illustrated in FIG. 7, a voltage is alternately applied to both of the positive side and the negative side with respect to the reference potential in a pulse waveform having the predetermined amplitude value Am and the predetermined pulse width Δtp. Then, in the example of FIG. 7, the pulse waveforms on the positive side and the negative side with respect to the reference potential are repeated within the predetermined cycle ΔT from the first round to the eighth round, and the ablation operation is repeated with the cycle ΔT as a unit. Note that the numerical ranges of the amplitude value Am, the pulse width Δtp, the time intervals between pulse waveforms Δtg1 and Δtg2, the cycle Δt, and the number of repetitions of the ablation operation in Example 2-1 illustrated in FIG. 7 are, for example, similar to the case of Example 1 (FIG. 5) described above, and the same applies to a case of Example 2-2 (FIG. 8) described below.

### A-2. Example 2-2

On the other hand, in the ablation method of Example 2-2 as well, for example, as illustrated in FIG. 8, basically similarly to the ablation method of Example 2-1, the power Pout is sequentially supplied to each of the plurality of electrode groups Ga to Ge. That is, in this example, in the order of (electrode group Ga→electrode group Gb→electrode group Gc→electrode group Gd→electrode group Ge), the power Pout is sequentially supplied to the electrodes 111 in each of the electrodes Ga to Ge, and the sequential supply of the power Pout is repeated.

However, in Example 2-2, unlike Example 2-1, when the power Pout is sequentially supplied to each of the plurality of electrode groups Ga to Ge, the power Pout is further sequentially supplied to a part of the electrodes 111 included in each of the electrodes Ga to Ge (each one of the electrodes 111 included in each of the electrode groups Ga to Ge in this example). Specifically, as illustrated in FIG. 6, for example, in the case of the four electrodes 111 (electrodes 111-1 to 111-4) included in each of the electrodes Ga to Ge, the following occurs. That is, for example, as indicated by the signs (1) to (4) in FIG. 8 for convenience, in each of the electrode groups Ga to Ge as well, the power Pout is sequentially supplied on each of the electrodes 111 in the order of (electrode 111-1→electrode 111-2→electrode 111-3→electrode 111-4), and this sequential supply of the power Pout is repeated.

In detail, for example, as illustrated in FIG. 8, basically similarly to the case of Example 2-1, a voltage is alternately applied to both of the positive side and negative side with respect to the reference potential in a pulse waveform having the predetermined amplitude value Am and the predetermined pulse width Δtp. Then, in the example of FIG. 8, the pulse waveforms on the positive side and the negative side with respect to the reference potential are repeated within the predetermined cycle ΔT from the first round to the eighth round, and the ablation operation is repeated with the cycle ΔT as a unit.

### B. Advantages and Effects

In the present modified example as well, basically, the same effects can be obtained by the same advantages as those of the embodiment. That is, in the present modified example as well, convenience can be improved during ablation on the affected area 90.

In particular, in the present modified example, the plurality of electrodes 111 are grouped into the plurality of electrode groups Ga to Ge, and when ablation is performed, the power Pout is sequentially supplied to each of the plurality of electrode groups Ga to Ge, and thus the following occurs. That is, for example, setting the appropriate electrode group according to, for example, the situation and the application by ablation allows enhancing effectiveness of ablation. As a result, convenience during ablation can be further improved in the present modified example.

Further, when the ablation is performed, in a case where the power Pout is sequentially supplied to each of the plurality of electrode groups Ga to Ge and the power Pout is further sequentially supplied to a part of (for example, one) the electrodes 111 included in each of the electrode groups Ga to Ge (equivalent to Example 2-2), the following occurs. In other words, for example, compared to the case (equivalent to Example 2-1) in which the power Pout is collectively supplied to at least a part of (for example, all of) the electrodes 111 included in each of the electrode groups Ga to Ge, the current during ablation concentratively flows to a part of the electrodes 111 in each of the electrodes Ga to Ge as well. This further increases the current density during ablation, and therefore the range of ablation on the affected area 90 further deepens. As a result, convenience during ablation can be more improved.

Note that, for example, the arrangement, the shape, and the number of (1 or a plurality of) the respective electrodes 111 close to the distal end of the shaft 11A (in the distal end proximal structure 6) are not limited to the examples described in the present modified example. Also, the shape of the distal end proximal structure 6 is not limited to the shape (the basket shape described above) described in the present modified example, and may be other shapes. Furthermore, the configuration of the distal end proximal structure 6 itself (for example, the arrangement, the shape, and the number in the branch points, the junctions, and the plurality of branch structures described above) are not limited to the configuration examples described in the present modified example, and may be other configurations.

In addition, although the configuration example of the electrode groups Ga to Ge has been specifically described in the present modified example, the configuration of the electrode group including the plurality of electrodes 111 is not limited to this example, and may be other configurations.

### 3. Other Modified Examples

Although the present disclosure has been described above with reference to the several embodiments, modified examples, and examples, the present disclosure is not limited to, for example, the embodiments, and various modifications are possible.

For example, in the above-described embodiment, the overall configuration of the ablation system has been specifically described, but it is not always necessary to include all of the devices, and other devices may be further included. Specifically, for example, in the above-described embodiment, the configuration of the ablation catheter (shaft) has been specifically described, but it is not always necessary to include all of the members, and other members may be further included. The configuration of the electrodes of the shaft (arrangement, shapes, numbers, and the like of the ring-shaped electrodes and the distal end electrode) is not limited to that mentioned in the above-described embodiment.

The ablation catheter described in, for example, the above-described embodiments may be an ablation catheter in which the vicinity of a distal end of a shaft can be bent in one direction or two directions according to an operation of an operating unit. Alternatively, it may be a fixed type ablation catheter in which a bending operation is not performed on the vicinity of a distal end of a shaft.

The values, ranges, magnitude relations, and the like of various parameters described in the above-described embodiment and the like are not limited to those described in the above-described embodiment, and may be other values, ranges, magnitude relations, and the like.

For example, in the above-described embodiment, the ablation catheter has been described as a specific example of the electromedical device, but the present disclosure is not limited to this example, and other electromedical devices may be applied.

For example, in the above-described embodiment, an example of a monopolar type in which ablation is performed by energization between the electrodes on the ablation catheter and the counter electrode plate has been described, but the present disclosure is not limited to this example. For example, in addition to between the electrodes on the ablation catheter and the counter electrode plate, ablation by energization may be performed between a plurality of electrodes on the ablation catheter.

For example, in the above-described embodiment, the ablation method (power supply method) has been described specifically, but the ablation method is not limited to the methods described in, for example, the above-described embodiment, and the ablation operation may be performed using another method.

For example, in the above-described embodiment, a case in which the target of ablation being the affected area having an arrhythmia inside the body of the patient or the affected area having the tumor as the examples, but the embodiment is not limited thereto. That is, the ablation system of the present disclosure is also applicable to the case where the target of ablation is another site inside the body of the patient (such as an organ or a body tissue).

The series of processes described in the above-described embodiment and the like may be performed by hardware (circuit) or software (program). When the series of processes are done by software, the software includes a group of programs for causing a computer to execute each function. Each program may be used by being preliminarily incorporated in the computer, for example, or may be installed and used in the computer from a network or a recording medium.

The various examples described so far may be applied in any combination.

Note that the effects described in the present specification are mere examples and effects of the present disclosure are not limited thereto. Other effects may be obtained.

The present disclosure may also have the following configuration.

(1)

A power supply device that includes a power supply unit and a control unit. The power supply unit supplies power to an electromedical device including a plurality of electrodes. The control unit controls the power supply unit such that the power is supplied to a part of the plurality of electrodes.

(2)

In the power supply device according to (1), the control unit controls the power supply unit such that the power is sequentially supplied to the part of the plurality of electrodes.

(3)

In the power supply device according to (2), the plurality of electrodes are grouped into a plurality of electrode groups. The control unit controls the power supply unit such that the power is sequentially supplied to each of the plurality of electrode groups.

(4)

In the power supply device according to (3), the control unit controls the power supply unit such that the power is collectively supplied to at least a part of electrodes included in the electrode group.

(5)

In the power supply device according to (4), the control unit controls the power supply unit such that the power is collectively supplied to all of electrodes included in the electrode group.

(6)

In the power supply device according to any one of (3) to (5), the electromedical device includes a shaft having a predetermined distal end proximal structure. The distal end proximal structure includes a branch point of the shaft, a junction positioned close to a most distal end of the shaft, and a plurality of branch structures that are portions to individually connect between the branch point and the junction in a curved shape and each of the plurality of branch structures includes the plurality of electrodes. The plurality of electrodes disposed in each of the branch structures constitute each of the electrode groups.

(7)

In the power supply device according to any one of (2) to (6), the control unit controls the power supply unit such that a sequential supply of the power to the part of the electrode is repeated.

(8)

A power supply method supplies power to an electromedical device including a plurality of electrodes. The power supply method includes: supplying the power to a part of the plurality of electrodes; and supplying the power to a remaining electrode among the plurality of electrodes.

### REFERENCE SIGNS LIST

1, 1A Ablation catheter
11, 11A Shaft
110 Distal end tip
111, 111a to 111d, 111-1 to 111-4 Electrode (ring-shaped electrode)
112 Electrode (distal end electrode)
12 Handle
121 Handle body
122 Rotating operating unit
3 Power Supply Device
31 Input unit
32 Power supply unit
33 Control unit
34 Display unit
4 Counter electrode plate
5 Ablation system
6 Distal end proximal structure
60 Deformation wire
61a to 61e Branch structure
9 Patient
90 Affected area
Pout Power
Vout Voltage
Am Amplitude value
CTL Control signal
It Temperature information
d Arrow
P1 Distal end
t Time
ΔT Cycle
Δtp Pulse width
Δtg1, Δtg2 Time interval
Ga to Ge Electrode group

1. A power supply device, comprising:
a power supply unit that supplies power to an electromedical device including a plurality of electrodes; and
a control unit that controls the power supply unit such that the power is supplied to a part of the plurality of electrodes.

2. The power supply device according to claim 1, wherein
the control unit controls the power supply unit such that the power is sequentially supplied to the part of the plurality of electrodes.

3. The power supply device according to claim 2, wherein
the plurality of electrodes are grouped into a plurality of electrode groups, and
the control unit controls the power supply unit such that the power is sequentially supplied to each of the plurality of electrode groups.

4. The power supply device according to claim 3, wherein
the control unit controls the power supply unit such that the power is collectively supplied to at least a part of electrodes included in the electrode group.

5. The power supply device according to claim 4, wherein
the control unit controls the power supply unit such that the power is collectively supplied to all of electrodes included in the electrode group.

6. The power supply device according to claim 3, wherein
the electromedical device includes a shaft having a predetermined distal end proximal structure,
the distal end proximal structure includes a branch point of the shaft, a junction positioned close to a most distal end of the shaft, and a plurality of branch structures that are portions to individually connect between the branch point and the junction in a curved shape, and each of the plurality of branch structures includes the plurality of electrodes, and
the plurality of electrodes disposed in each of the branch structures constitute each of the electrode groups.

7. The power supply device according to claim 2, wherein
the control unit controls the power supply unit such that a sequential supply of the power to the part of the electrode is repeated.

8. A power supply method that supplies power to an electromedical device including a plurality of electrodes, the power supply method comprising:
supplying the power to a part of the plurality of electrodes; and
supplying the power to a remaining electrode among the plurality of electrodes.
